(19) United States
(12) Patent Application Publication
(10) Pub. No.: US 2017/0238858 A1
(43) Pub. Date: Aug. 24, 2017

(54) DEPRESSION ASSESSMENT SYSTEM AND DEPRESSION ASSESSMENT METHOD BASED ON PHYSIOLOGICAL INFORMATION

(71) Applicants: SOUTH CHINA UNIVERSITY OF TECHNOLOGY, Guangzhou City (CN); SHENZHEN SAYES MEDICAL TECHNOLOGY CO., LTD., Shenzhen City (CN)

(72) Inventors: Rongqian Yang, Guangzhou City (CN); Xiuwen Chen, Guangzhou City (CN); Ruixue Lv, Shenzhen City (CN); Chuanxu Song, Shenzhen City (CN)

(21) Appl. No.: 15/109,815

(22) PCT Filed: Oct. 29, 2015
(86) PCT No.: PCT/CN2015/093158

§ 371 Date: Jul. 6, 2016

(30) Foreign Application Priority Data
Jul. 30, 2015 (CN) 201510468922.X

(51) Int. Cl. A61B 5/16; A61B 5/024; A61B 5/0476; A61B 5/0456; A61B 5/0496; A61B 5/00; A61B 5/01; A61B 5/04; A61B 5/0402; A61B 5/0488

(52) U.S. Cl. CPC A61B 5/165; A61B 5/0402; A61B 5/02416; A61B 5/0476; A61B 5/04884; A61B 5/0496; A61B 5/4812; A61B 5/01; A61B 5/0022; A61B 5/04012; A61B 5/0456; A61B 5/742; A61B 5/0533

(57) ABSTRACT

The present invention discloses a depression assessment system based on physiological information, comprising an information acquisition module, a signal processing module, a parameters calculation module, a feature selection module, a machine learning module and an output result module. The present invention further discloses a depression assessment method based on various physiological information, comprising the following steps: 1, processing electrocardiogram (ECG) signal and one or more of photoplethysmography (PPG) signal, electroencephalogram (EEG) signal, galvanic skin response (GSR)signal, electrogastrography (EGG) signal, electromyogram (EMG) signal, electrooculogram (EOG) signal, polysomnogram (PSG) signal and temperature signal, and calculating signal parameters; 2, normalizing the obtained signal parameters, and performing the feature selection on parameters set formed by the normalized signal parameters to obtain feature parameters set; and 3, performing machine learning by utilizing the obtained feature parameters set, and establishing a depression assessment mathematic model to assess the depression level by utilizing a relationship between the feature parameters set and the depression level. The present invention has the advantage that the subjectivity of the assessment by utilizing the depression rating scale can be avoided.

### CROSS REFERENCE TO RELATED APPLICATION(S)

This application is a national stage filing under 35 U.S.C. 371 of International Application No. PCT/CN2015/093158, filed Oct. 29, 2015, which claims priority to CN2015/10468922.X, filed Jul. 30, 2015, the disclosures of which are incorporated herein by reference.

### TECHNICAL FIELD

The present invention relates to a depression assessment technology, in particular to a depression assessment system and a depression assessment method based on physiological information.

### BACKGROUND

As the development of the society, people will face increasing pressure, and the incidence rate of depression will be higher and higher. It is investigated that there are about 90 million depression patients in China accounting for 6.4% of the total population. There are about 350 million depression patients in the whole world. The depression patient is generally in a blue mood, loses the interests in things once he is interested in and is lower in attention. The depression is classified into a light level, a medium level and a severe level, and the patient with severe syndrome has a suicidal tendency. The cause of the depression is complicated and is not single, biological factors, psychological factors and social factors collectively form a biology-psychology-society uniform mode, and the depression is influenced by the factors such as genetic factors, biochemical factors, neuroendocrine factors, psychosocial factors, etc. The research on the pathogenesis of the depression is generally concentrated on neurotransmitters and their acceptors, and particularly on monoamine neurotransmitters and their acceptors, and the research suggest that neuropeptides play an important role in the incidence of the depression. However, so far, the pathogenesis of the depression has no uniform final conclusion.

At present, the depression is clinically assessed mainly according to the medical history, clinical symptoms, etc. The depression is generally assessed according to the assessment standards such as ICD-10 and DSM-IV in the world. In China, ICD-10 is mainly adopted to assess the depression. Whether a subject has depression or not is judged by the depression symptoms and a Self-rating Depression Scale (SDS). Such assessment way may be affected by the subjective description of the subject and the subjective factor and clinical experience of a psychologist and is not an effective method for objectively assessing the depression. Therefore, a method for assessing the depression on the basis of the physiological information is required to objectively quantify whether the subject suffers from depression and to quantify the depression level of the subject.

According to the previous research, the physiological information of the depression patient such as electrocardiogram (ECG), photoplethysmography (PPG), electroencephalogram (EEG), galvanic skin response (GSR), electrogastrography (EGG), electromyogram (EMG), electrooculogram (EOG), polysomnogram (PSG) and temperature, etc. are different from that of a normal person. The differences are reflected on the aspects such as time domain, frequency domain and time domain geometric parameters, etc. of electrical signals. Therefore, there are a research foundation, feasibility and clinical practicability to process the signal, to calculate a great amount of signal parameters and to establish a depression assessment mathematic model for assessing the depression according to the differences of various physiological information performances.

### SUMMARY

A primary object of the present invention lies in overcoming the weaknesses and defects of the existing depression assessment technology, and providing a depression assessment system based on the physiological information. The depression assessment system calculates parameters of the physiological information such as time domain, frequency domain etc. by acquiring ECG, and one or more physiological information of PPG, EEG, GSR, EGG, EMG, EOG, PSG and temperature, extracts a feature parameters set and establishes a depression assessment mathematic model to further assess whether the subject suffers from depression and to assess the depression level.

Another object of the present invention lies in overcoming the weaknesses and defects of the existing depression assessment method, and providing an assessment method applied to the depression assessment system based on the physiological information. The assessment method can objectively quantitatively assess whether the subject suffers from depression and assess the depression level.

The primary object of the present invention is realized by the following technical solution: a depression assessment system based on the physiological information comprises an information acquisition module, a signal processing module, a parameters calculation module, a feature selection module, a machine learning module and an output result module.

The information acquisition module is used for acquiring ECG signal and selectively acquiring one or more of PPG signal, EEG signal, GSR signal, EGG signal, EMG signal, EOG signal, PSG signal and temperature signal. The signal acquired by the information acquisition module is transmitted in a wire transmission manner by a USB serial port or transmitted in a Bluetooth wireless transmission manner to the signal processing module.

The signal processing module is used for performing the signal processing on the physiological information and comprises an ECG signal processing unit, an PPG signal processing unit, an EEG signal processing unit, an GSR signal processing unit, an EGG signal processing unit, an EMG signal processing unit, an EOG signal processing unit, an PSG signal processing unit and a temperature signal processing unit, wherein the ECG signal processing unit is used for performing baseline removal processing, filtering de-noising processing, sinus beat extraction intervals (RR intervals) processing, interpolation processing, Fourier transformation processing as well as spectral analysis and spectral estimation processing. The PPG signal processing unit is used for performing baseline removal processing, filtering de-noising processing, pulse extraction intervals (PP intervals) processing, interpolation processing, Fourier transformation processing as well as spectral analysis and spectral estimation processing. The EEG signal processing unit is used for performing baseline removal processing, threshold value de-noising processing, wavelet decomposition processing as well as spectral analysis and spectral estimation processing. The GSR signal processing unit is used for performing baseline removal processing and wavelet filtering processing. The EGG signal processing unit is used for performing baseline removal processing, Hilbert-Huang transformation processing, wavelet analysis processing, multi-resolution analysis processing and independent component analysis processing. The EMG signal processing unit is used for performing baseline removal processing and wavelet packet self-adaptive threshold value processing. The EOG signal processing unit is used for performing baseline removal processing, weighting median filtering processing and wavelet transformation processing. The PSG signal processing unit is used for processing sleep EEG signal, sleep EMG signal and sleep EOG signal, for performing the baseline removal processing, the threshold value de-noising processing, the wavelet analysis processing as well as the spectral analysis and the spectral estimation processing on the sleep EEG signal, for performing the baseline removal processing, the weighting median filtering processing and the wavelet transformation processing on the sleep EOG signal, and performing the baseline removal processing, the wavelet packet self-adaptive threshold value de-noising processing and the sleep staging processing on the sleep EMG signal. The temperature signal processing unit is used for performing the baseline removal processing, the threshold value filtering processing, and the establishment of a relational expression between a temperature value and an image gray value. The signal processing module outputs a processed signal to the parameters calculation module.

The parameters calculation module is used for calculating the signal parameters of the processed signal and comprises an ECG parameters calculation unit, an PPG parameters calculation unit, an EEG parameters calculation unit, an GSR parameters calculation unit, a EGG parameters calculation unit, an EMG parameters calculation unit, an EOG parameters calculation unit, an PSG parameters calculation unit and a temperature parameters calculation unit, wherein the ECG parameters calculation unit is used for calculating RR intervals, mean value of all RR intervals, standard deviation of NN intervals (SDNN) of heartbeat intervals, root mean square of successive difference(RMSSD) of successive heartbeats, percentage of normal-to-normal interval more than 50 ms(PNN50) of successive heartbeats, standard deviation of successive differences (SDSD) of heartbeats, very low frequency (VLF)power, low frequency (LF)power, high frequency (HF)power, total power (TP), ratio of the low frequency power to the high frequency power(LF/HF), standard deviation (SD1) perpendicular to y=x in RR intervals scatter diagram, standard deviation (SD2) of a y=x straight line in the RR intervals scatter diagram, slope (a1) of the short-term detrended fluctuation analysis and slope (a2) of the long-term detrended fluctuation analysis. The PPG parameters calculation unit is used for calculating PP intervals, mean value of all PP intervals, standard deviation of NN intervals (SDNN) of pulse intervals, root mean square of successive difference (RMSSD) of successive pulses, percentage of normal-to-normal interval more than 50 ms(PNN50) of successive pulses, standard deviation of successive differences (SDSD) of pulses, very low frequency (VLF)power, low frequency (LF)power, high frequency (HF)power, total power (TP), ratio of the low frequency power to the high frequency (LF/HF) power, standard deviation (SD1) perpendicular to y=x in PP interval scatter diagram, standard deviation (SD2) of a y=x straight line in the PP interval scatter diagram, slope (a1) of the short-term detrended fluctuation analysis and slope (a2) of the long-term detrended fluctuation analysis. The EEG parameters calculation unit is used for calculating δ wave amplitude, δ wave power, δ wave mean value, δ wave variance, δ wave deviation degree, δ wave kurtosis, θ wave amplitude, θ wave power, θ wave mean value, θ wave variance, θ wave deviation degree, θ wave kurtosis, α wave amplitude, wave power, α wave mean value, α wave variance, α wave deviation degree, α wave kurtosis, β wave amplitude, β wave power, β wave mean value, β wave variance, β wave deviation, β wave kurtosis and wavelet entropy. The GSR parameters calculation unit is used for calculating a sympathetic skin response latency, a sympathetic skin response wave amplitude and a skin resistance value. The EGG parameters calculation unit is used for calculating normogastria, a slow wave, a bradygastria component and a tachygastria component. The EMG parameters calculation unit is used for calculating a basic value, a minimum value, a highest value, an EMG decreasing capacity and an EMG curve. The EOG parameters calculation unit is used for calculating R wave component, r wave component, S wave component and s wave component. The PSG signal parameters calculation unit is used for calculating sleep latency, total sleep time, arousal index, shallow sleep period (S1), light sleep period (S2), middle sleep period (S3), deep sleep period (S4), rapid eye movement(REM) sleep percentage, REM sleep cycles, REM sleep latency, REM sleep intensity, REM sleep density and REM sleep time. The temperature parameters calculation unit is used for calculating the temperature distribution in a human body. The parameters calculation module outputs the signal parameters to the feature selection module.

The feature selection module is used for acquiring the feature parameters set related to the depression level from all signal parameters. The feature selection module outputs the feature parameters set to the machine learning module.

The machine learning module is used for training a depression level quantification classifier and utilizing the feature parameters set to establish the depression assessment mathematic model to quantify the depression level. The machine learning module outputs the depression level to the output result module.

The output result module is used for displaying the depression level outputted by the depression assessment mathematic model.

Another object of the present invention is realized by means of the following technical solution: the assessment method applied to the depression assessment system based on the physiological information can comprise the following steps:

step 1: acquiring the physiological information; the physiological information including ECG information and one or more information of PPG, EEG, GSR, EGG, EMG, EOG, PSG and temperature;

step 2: processing the acquired signals such as the ECG signal and one or more of the PPG signal, the EEG signal, the GSR signal, the EGG signal, the EMG signal, the EOG signal, the PSG signal and the temperature signal,

step 3: calculating the processed signal to obtain signal parameters;

step 4: normalizing the calculated signal parameters, and performing the feature selection on parameters set formed by the normalized signal parameters to obtain feature parameters set;

the feature selection is divided into a feature search portion and an evaluation criteria portion, wherein the search algorithm adopts one of or a combination of more than one of the following algorithms: a complete search algorithm, a sequential search algorithm, a random search algorithm, a genetic algorithm, a simulated annealing algorithm and a traceable greedy search expansion algorithm; and the evaluation criteria selectively utilizes a wapper model or a CfsSubsetEval attribute evaluation method. During the evaluation process, the ECG signal and the PPG signal are acquired, and the feature selection adopts a way combining the complete search algorithm and the wapper model; and during the evaluation process, the ECG signal, the GSR signal and the PSG signal are acquired, and the feature selection adopts a way combining the random search algorithm and the CfsSubsetEval attribute evaluation method. The appropriate algorithm combination with high accuracy is selected according to different types of the acquired signals.

step 5: performing the machine learning by utilizing the feature parameters set obtained in step 4, establishing a depression assessment mathematic model by utilizing the relationship between the feature parameters set and the depression level, outputting a depression level assessment result by utilizing the depression assessment mathematic model, and assessing the depression level according to the depression level assessment result;

the machine learning being used for training the depression assessment mathematic model, establishing the depression assessment mathematic model by utilizing the feature parameters set during the machine learning process, and utilizing one of or a combination of more than one of the following algorithms for the machine learning algorithm: bayes classifier, decision tree algorithm, AdaBoost algorithm, k-nearest-neighbor algorithm and support vector machine; expression of the depression assessment mathematic model is as follows:

$$Y = {\sum }_{i = 1}^{n} {a}_{i} {y}_{i}$$

wherein, Y is an output value of the depression assessment mathematic model, n is the number of selected machine learning algorithm, Yᵢ is output value of the ith algorithm, aᵢ is coefficient of the ith algorithm, and i is positive integer;

step 6: inputting the result of depression level assessment of the step 5 into the output result module.

In the step 4, the normalization method is as follows:

$${X}_{in} = \frac{{X}_{i} - {X}_{imean}}{{X}_{istd}}$$

wherein, X refers to signal parameter of the parameter set; Xᵢ indicates the ith normalized signal parameter value, Xᵢₙ indicates the ith normalized value, Xₘₑₐₙ indicates normal mean value of the ith parameter, X_{istd} indicates normal standard difference of the ith parameter, and i is positive integer. After the depression assessment mathematic model based on various physiological information is established, the depression level is evaluated by utilizing the output result of the depression assessment mathematic model, and the depression level is divided into five classes: normal, common, light depression, moderate depression and severe depression.

In the step 2, the signal processing includes the ECG signal processing, the PPG signal processing, the EEG signal processing, the GSR signal processing, the EGG signal processing, the EMG signal processing, the EOG signal processing, the PSG signal processing and the temperature signal processing; the ECG signal processing includes the baseline removal processing, the filtering de-noising processing, the RR intervals extraction, the interpolation processing, the Fourier transformation processing as well as the spectral analysis and spectral estimation processing; the EEG signal processing includes the baseline removal processing, the threshold value de-noising processing, the wavelet decomposition processing as well as the spectral analysis and spectral estimation processing; the GSR signal processing includes the baseline removal processing and the wavelet filtering processing; the EGG signal processing includes the baseline removal processing, the Hilbert-Huang transformation processing, the wavelet analysis, the multi-resolution analysis and the independent component analysis; the EMG signal processing includes the baseline removal processing and the wavelet packet self-adaptive threshold value de-noising processing; the EOG signal processing includes the baseline removal processing, the weighting median filtering processing and the wavelet transformation processing; the PSG signal processing includes the processing of the sleep EEG signal, the sleep EMG signal and the sleep EOG signal; the baseline removal processing, the threshold value de-noising processing, the wavelet decomposition processing as well as the spectral analysis and spectral estimation processing are conducted on the sleep EEG signal; the baseline removal processing, the weighted median filtering processing and the wavelet transformation processing are conducted on the sleep EOG signal; the baseline removal processing, the wavelet packet self-adaptive threshold value de-noising processing and the sleep staging processing are conducted on the sleep EMG signal; and the temperature signal processing includes the baseline removal processing, the threshold value filtering processing and the establishment of a relational expression between the temperature value and the image gray value.

In the step 3, the calculation of signal parameters of the processed signal includes the ECG parameters calculation, the PPG parameters calculation, the EEG parameters calculation, the GSR parameters calculation, the EGG parameters calculation, the EMG parameters calculation, the EOG parameters calculation, the PSG parameters calculation and the temperature parameters calculation; the ECG parameters calculation includes the calculation of the RR intervals, the time-domain parameters, the frequency-domain parameters and the time-domain geometric parameters; the time-domain parameters include mean value, SDNN, RMSSD, PNN50 and SDSD; the frequency-domain parameters include VLF, LF, HF, TP and LF/HF; the time-domain geometric parameters include SD1, SD2, a1 and a2; the PPG parameters calculation includes the calculation of the PP intervals, the time-domain parameters; the time-domain parameters include mean value, SDNN, RMSSD, PNN50 and SDSD; the frequency-domain parameters include VLF, LF, HF, TP and LF/HF; the time-domain geometric parameters include SD1, SD2, a1 and a2; the EEG parameters calculation includes the calculation of δ wave amplitude, δ wave power, δ wave mean value, δ wave variance, δ wave deviation degree, δ wave kurtosis, θ wave amplitude, θ wave power, θ wave mean value, θ wave variance, θ wave deviation, θ wave kurtosis, α wave amplitude, α wave power, α wave mean value, α wave variance, α deviation degree, α wave kurtosis, β wave amplitude, β wave power, β wave mean value, β wave variance, β wave deviation degree, β wave kurtosis and wavelet entropy; the GSR parameters calculation includes the calculation of sympathetic skin response latency, the sympathetic skin response amplitude and the skin resistance value; the EGG parameters calculation includes the calculation of normogastria, the slow wave, the bradygastria and tachygastria components; the EMG parameters calculation includes the calculation of basic value, the minimum value, the highest value, the EMG decreasing capacity and the EMG curve; the EOG parameters calculation includes the calculation of R wave, r wave, S wave and s wave components; the PSG sleep signal parameters calculation includes the calculation of the sleep latency, the total sleep time, the arousal index, S1, S2, S3, S4, the REM sleep percentage, the REM sleep cycles, the REM sleep latency, the REM sleep intensity, the REM sleep density and the REM sleep time; and the temperature parameters calculation includes the calculation of the temperature distribution in the human body.

In the step 4, the feature selection trains a data set according to all signal parameters outputted by the parameters calculation module, each sample is represented by a feature set, and a feature sub-set is generated; an optimum feature subset in the feature set is acquired in a searching manner according to the evaluation criteria; the current feature subsets are compared and evaluated; when the acquired feature subset is the optimum feature subset, a termination condition is satisfied, and the feature parameters set related to the depression level is outputted; the search algorithm adopts one of or a combination of more than one of the following algorithms: the complete search algorithm, the sequential search algorithm, the random search algorithm, the genetic algorithm, the simulated annealing search algorithm and the traceable greedy search expansion algorithm; and the evaluation criteria adopts one of or a combination of two of the following algorithms: the wapper model and the CfsSubsetEval attribute assessment method.

Compared to the prior art, the present invention has the following advantages and beneficial effects:

1. the establishment of the depression assessment mathematic model has a research foundation; the parameters of the ECG signal, the PPG signal, the EEG signal, the GSR signal, the EGG signal, the EMG signal, the EOG signal, the PSG signal and the temperature signal are associated with the depression, therefore, it is feasible to assess the depression level by utilizing the output result of the depression assessment mathematic model based on the physiological information;

2. the depression level is objectively quantified by utilizing the assessment way of the depression assessment data model by physiological parameters, thereby improving the traditional depression assessment level way, avoiding the subjectivity of the assessment of the level, satisfying the clinical demand and having the clinical practicability;

3. the depression is assessed in combination with the physiological parameters such as the ECG, the PPG, the EEG, the GSR, the EGG, the EMG, the EOG, the PSG and the temperature, thereby enriching the cross research methods of the neurosciences field and the psychology field;

4. the present invention carries out the signal processing, parameters calculation and mathematic modeling on one of or a combination of more than one of the ECG signal, the PPG signal, the EEG signal, the GSR signal, the EGG signal, the EMG signal, the EOG signal, the PSG signal and the temperature signal; the combination of a plurality of signals can be selected for the assessment, thereby having the flexibility and novelty;

5. the present invention provides the method for normalizing the signal parameters; the parameters are compared with the mean value and the standard deviation in a normal sample, and the difference of the parameters on the aspect of the numerical value and the deviation is eliminated, so that the feature selection of the parameter set is more scientific and more effective; and

6. The present invention proposes the algorithm combination of various feature selections and the machine learning, so that the establishment of mathematic model is more flexible according to different types of signals.

### BRIEF DESCRIPTION OF THE DRAWINGS

FIG. 1 is a schematic diagram of the depression assessment system based on the physiological information.

FIG. 2 is a structural diagram of the depression assessment system based on the physiological information.

### DETAILED DESCRIPTION

The present invention is further described below in details in conjunction with embodiments and drawings, but the present invention is not limited to the following embodiments. Embodiments

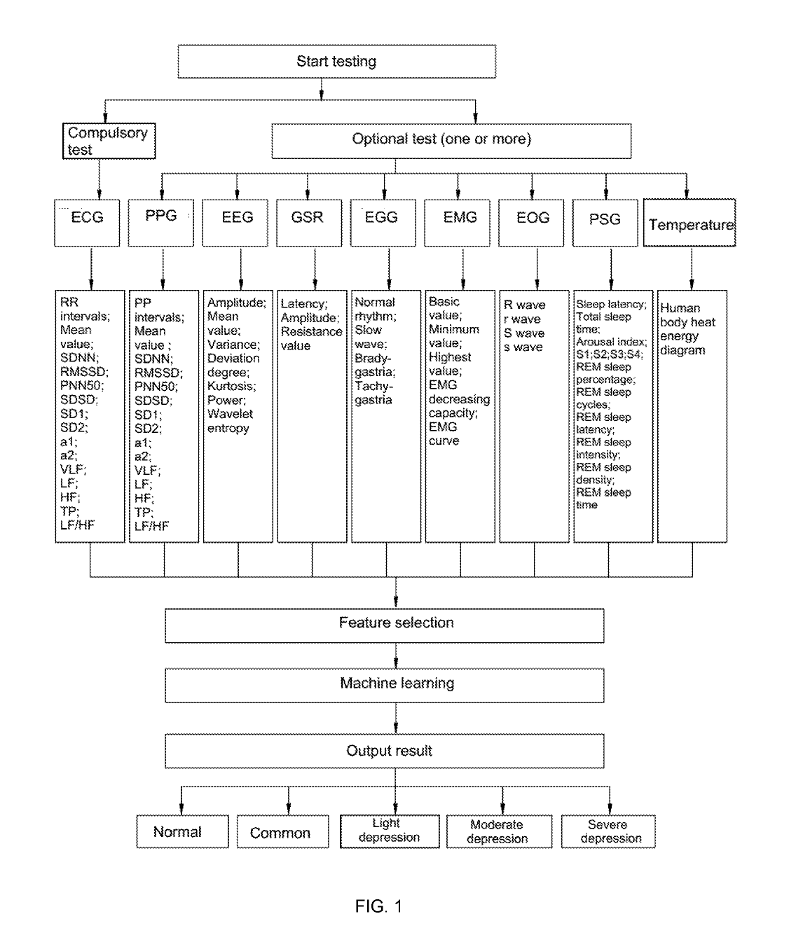

As shown in FIG. 1, the depression assessment system based on physiological information comprises an information acquisition module, a signal processing module, a parameters calculation module, a feature selection module, a machine learning module and an output result module; and a signal acquired by the information acquisition module is transmitted in a wire transmission manner by a USB serial port or transmitted to the signal processing module in a Bluetooth wireless transmission manner. The signal processing module outputs a processed signal to the parameters calculation module. The parameters calculation module outputs the signal parameters to the feature selection module. The feature selection module outputs a feature parameters set to the machine learning module. The machine learning module outputs the depression level to the output result module.

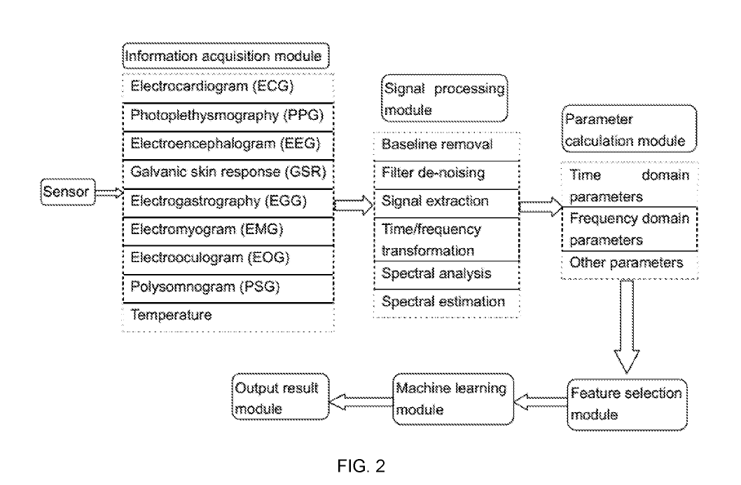

The structure of the depression assessment system based on the physiological information is as shown in FIG. 2; the information acquisition module is used for acquiring ECG signal and acquiring one or more of PPG signal, EEG signal, GSR signal, EGG signal, EMG signal, EOG signal, PSG signal and temperature signal. The signal processing module is used for processing the physiological information including the baseline removal processing, the filtering de-noising processing, the heartbeat intervals extraction processing, the time/frequency transformation processing as well as the spectral analysis and spectral estimation processing. The parameters calculation module is used for calculating the signal parameters of the processed signal including the time-domain parameters, the frequency-domain parameters and the time-domain geometric parameters of the heat rate variability, and for selectively calculating the time-domain parameters, the frequency-domain parameters, the histogram parameters and the distribution diagram parameters of one or more of the PPG signal, the EEG signal, the GSR signal, the EGG signal, the EMG signal, the EOG signal, the PSG signal and the temperature signal according to the acquired physiological information. The feature selection module is used for acquiring the feature parameters set related to the depression level from all signal parameters. The machine learning module is used for training a depression level quantification classifier and utilizing the feature parameters set to establish the depression assessment mathematic model to quantify the depression level. The output result module is used for displaying the depression level outputted by the depression assessment mathematic model.

The depression assessment method based on various physiological information of the system comprises the following steps:

step 1: acquiring the physiological information, wherein the physiological information includes the ECG information and one or more information of the PPG, EEG, GSR, EGG, EMG, EOG, PSG and temperature, wherein:

the ECG signal acquisition can selectively measure ECG signal at a five-minute still state, and the sampling rate for the ECG acquisition can select 500 Hz or greater than 500 Hz;

the PPG acquisition selectively utilizes pulse signal acquired by a pulse sensor, reflecting the volume variation at the end of a blood vessel outputted from an infrared transmission point part or utilizes a vibration-type measurement method to acquire wrist pulse signal; and the sampling rate for acquiring the PPG can select 500 Hz or greater than 500 Hz;

the EEG acquisition selectively adopts 10 to 20 systematic points to excite and acquire the spontaneous EEG activity of cerebral cortex;

the GSR acquisition adopts the sympathetic skin response test, and the single pulse transcutaneous electrical stimulation is performed on the nerves in the middle of the wrist to test the sympathetic skin response starting latency and amplitude as well as to test the skin resistance value at the thenar eminence of a right hand and at forearm dorsal;

the EGG acquisition adopts a body surface electrode placed on the midsection to measure the gastric EMG activity;

the EMG acquisition adopts the stimulation of biological feedback instrument, and an EMG electrode connected to the forehead measures the EMG signal;

the EOG acquisition adopts the measurement of the closed eye movement (CEM);

the PSG acquisition adopts a way of simultaneously acquiring the EOG, the underjaw EMG and the EEG to measure the sleep time and parameters thereof;

the temperature acquisition can adopt a way for measuring the temperature in the human body by adopting an infrared temperature measuring principle. The signal acquisition belongs to the conventional signal acquisition.

In the step 2: the physiological information acquired in the step 1 is processed, and the signal parameters are calculated; the specific parameters are shown in the following table 1, and table 1 is a description table of electrical signals and parameters thereof:

wherein, the ECG signal processing and the parameters calculation calculate the RR intervals, mean value, SDNN, RMSSD, PNN50, SDSD, VLF, LF, HF, TP, LF/HF, SD1, SD2, a1 and a2 by means of the baseline removal processing, the filtering de-noising processing, the RR intervals extraction processing, the interpolation processing, the Fourier transformation processing as well as the spectral analysis and the spectral estimation processing;

the PPG signal processing and the parameters calculation adopt the baseline removal processing, the filtering de-noising processing, the pulse extraction intervals (PP intervals) processing, the interpolation processing, the Fourier transformation processing as well as the spectral analysis and the spectral estimation processing on the PPG signal;

the EEG signal processing and the parameters calculation adopt the baseline removal processing, the threshold value de-noising processing, the wavelet decomposition processing as well as the spectral analysis and the spectral estimation processing on the EEG signal to calculate the δ wave amplitude, the δ wave power, the δ wave mean value, the δ wave variance, the δ wave deviation degree, the δ wave kurtosis, the θ wave amplitude, the θ wave power, the θ wave mean value, the θ wave variance, the θ wave deviation degree, the θ wave kurtosis, the α wave amplitude, the α wave power, the α wave mean value, the α wave variance, the α wave deviation degree, the α wave kurtosis, the β wave amplitude, the β wave power, the β wave mean value, the β wave variance, the β wave deviation degree, the β wave kurtosis and the wavelet entropy;

the GSR signal processing and the parameters calculation adopt the baseline removal processing and the wavelet filtering on the GSR signal to calculate the sympathetic skin response latency, the sympathetic skin response wave amplitude and the skin resistance value;

the EGG signal processing and the parameters calculation adopt the baseline removal processing, the Hilbert-Huang transformation processing, the wavelet analysis processing, the multi-resolution analysis processing and the independent component analysis processing on the EGG signal to calculate the normogastria, the slow waves, the Bradygastria component and the tachygastria component;

the EMG signal processing and the parameters calculation adopt the baseline removal processing and the wavelet packet self-adaptive threshold value de-noising processing on the EMG signal to calculate the basic value, the minimum value, the highest value, the EMG decreasing capacity and the EMG curve;

the EOG signal processing and the parameters calculation adopt the baseline removal processing, the weighting median filtering processing and the wavelet transformation processing on the EOG signal to calculate the R wave component, the r wave component, the S wave component and the s wave component;

the PSG signal processing and the parameters calculation adopt the baseline removal processing, the threshold value de-noising processing, the wavelet decomposition analysis processing as well as the spectral analysis and the spectral estimation processing on the sleep EEG signal, adopt the baseline removal processing, the weighting median filtering processing and the wavelet transformation processing on the sleep EOG signal and adopt the baseline removal processing, the wavelet packet self-adaptive threshold value de-noising processing and the sleep staging processing on the sleep EMG signal to calculate the sleep latency, the total sleep time, the arousal index, S1, S2, S3, S4, the REM sleep percentage, the REM sleep cycles, the REM sleep latency, the REM sleep intensity, the REM sleep density and the REM sleep time; and

the temperature signal processing and the parameters calculation adopt the baseline removal processing, the threshold value filtering processing, and the establishment of a relational expression between a temperature value and an image gray value on the temperature signal to calculate the temperature distribution in the human body.

TABLE 1
Electrical signals and parameters thereof

| | | | Number of |
|---|---|---|---|
| Signal | Parameter | Description | parameters |
| ECG | RR intervals | Sinus heartbeat interval, RR intervals | 1 |
| PPG | PP intervals | PPG adjacent P wave interval | 1 |
| ECG/PPG | Mean value, | the mean time of all RR intervals; the standard | 5 |
| | SDNN, RMSSD, | deviation of heartbeat intervals; the root mean | |
| | PNN50, SDSD | square of successive difference of successive | |
| | | heartbeats, percentage of normal-to-normal | |
| | | interval more than 50 ms, standard deviation of | |
| | | successive differences of heartbeats | |
| ECG/PPG | VLF, LF, HF, TP, | the very low frequency power: 0.003 Hz- | 5 |
| | LF/HF | 0.04 Hz; the low frequency power: 0.04 Hz- | |
| | | 0.15 Hz; the high frequency power: 0.15 Hz- | |
| | | 0.4 Hz; the frequency total power: | |
| | | VLF + LF + HF; the ratio of the low frequency | |
| | | power to the high frequency power | |
| ECG/PPG | SD1, SD2, a1, a2 | the standard deviation perpendicular to y = x in | 4 |
| | | the RR interval scatter diagram; the standard | |
| | | deviation of the y = x straight line in the RR | |
| | | interval scatter diagram; the slope of the short- | |
| | | term detrended fluctuation analysis; slope of | |
| | | long-term detrended fluctuation analysis | |
| EEG | δ wave, θ wave, α | the frequency of δ waves is 0.5 Hz-4 Hz; the | 4 |
| | wave and β wave | frequency of θ waves is 4 Hz-8 Hz; the | |
| | amplitudes | frequency of α waves is 8 Hz-14 Hz; and the | |
| | | frequency of β waves is 14 Hz-30 Hz. | |
| EEG | the mean value, | the mean value, the variance, the deviation | 4 |
| | the variance, the | degree and the kurtosis of the amplitude are | |
| | deviation degree, | extracted from the EEG histogram. | |
| | the kurtosis | | |
| EEG | δ wave, θ wave, α | δ wave, θ wave, α wave and β wave power at a | 4 |
| | wave and β wave | power spectral frequency waveband. | |
| | power | | |
| EEG | wavelet entropy | wavelet transformation spectral entropy | 1 |
| GSR | sympathetic skin | conduction time interval of sudomotor | 1 |
| | response latency | impulsion in a whole reflex arc | |
| GSR | sympathetic skin | skin reflectivity potential amplitude | 1 |
| | response wave | | |
| | amplitude | | |
| GSR | skin resistance | skin resistance value at thenar eminence of a | 1 |
| | value | right hand and forearm dorsal. | |
| EGG | normogastria | main frequency (DF): 2.4 cycles/min-3.6 | 1 |
| | | cycles/min | |
| EGG | slow wave | The electrical activity varied periodically on | 1 |
| | | the gastric wall. | |
| EGG | bradygastria | Bradygastria: 0.5 cycles/min-2.4 cycles/min | 1 |
| EGG | tachygastria | tachygastria: 3.7 cycles/min-9.0 cycles/min | 1 |
| EMG | the basic value, | the mean value of the EMG potential at the still | 3 |
| | the minimum | state; the minimum value of the EMG potential | |
| | value, the highest | at the still state; and the highest value of the | |
| | value | EMG potential at the still state | |
| EMG | EMG decreasing | the ratio of the difference value between the | 1 |
| | capacity | basic value and the minimum value in the basic | |
| | | value | |
| EMG | EMG curve | the curve of the EMG potential varied along | 1 |
| | | the time at the still state | |
| EOG | R wave | R wave: the rectangular waves of the rapid | 4 |
| | r wave | closed eye movement, and the amplitude ≧3°; r | |
| | S wave | wave: the rectangular waves of the rapid closed | |
| | s wave | eye movement, and the amplitude is 1°-3°; S | |
| | | wave: single-peak or sinusoidal waves of the | |
| | | slow closed eye movement, and the | |
| | | amplitude ≧7°; s wave: the single-peak or | |
| | | sinusoidal waves of the slow closed eye | |
| | | movement, and the amplitude is 3°-7°. | |
| PSG | sleep latency, total | first stage sleep from the moment when the | 3 |
| | sleep time, arousal | light is turned off to the moment when a first | |
| | index | non-rapid eye movement sleep with the | |
| | | duration of 3 minutes; total time of all non- | |
| | | rapid eye movement sleep and the non-rapid | |
| | | eye movement sleep; the average arousal times | |
| | | per hour, and the arousal index = total arousal | |
| | | times/total sleep time. | |
| PSG | S1, S2, S3, S4 | shallow sleep period; light sleep period; | 4 |
| | | middle sleep period; deep sleep period | |
| PSG | REM sleep | the percentage of the REM sleep time in the | 1 |
| | percentage | total sleep time | |
| PSG | REM sleep | the times of the REM sleep during the sleep | 5 |
| | cycles; REM | process; the time from the moment when the | |
| | sleep latency; | sleep is onset to the moment when a first REM | |
| | REM sleep | sleep occurs; the REM intensity; the REM | |
| | intensity; REM | density; the total time of the REM sleep | |
| | sleep density and | | |
| | REM sleep time | | |
| temperature | the heat energy | The distribution diagram of temperature in the | 1 |
| | diagram of the | human body | |
| | human body | | |

step 3: calculating the processed signal to obtain signal parameters;

step 4: normalizing the calculated signal parameters obtained in step 3, and performing the feature selection on parameters set formed by the normalized signal parameters to obtain feature parameters set, wherein the normalizing method is:

$${X}_{in} = \frac{{X}_{i} - {X}_{imean}}{{X}_{istd}} ,$$

wherein, X refers to signal parameter of the parameter set; Xᵢ indicates the i^{th} normalized signal parameter value, Xᵢₙ indicates the i^{th} normalized value, X ᵢₘₑₐₙ indicates normal mean value of the i^{th} parameter, X_{istd} indicates a normal standard difference of the i^{th} parameter, and i is positive integer. The feature selection is divided into a feature search portion and an evaluation criteria portion, wherein the search algorithm adopts one of or a combination of more than one of the following algorithms: a complete search algorithm, a sequential search algorithm, a random search algorithm, a genetic algorithm, a simulated annealing algorithm and a traceable greedy search expansion algorithm; and the evaluation criteria selectively utilizes a wapper model or a CfsSubsetEval attribute evaluation method. During the evaluation process, the ECG signal and the PPG signal are acquired, and the feature selection adopts a way combining the complete search algorithm and the wapper model; and during the evaluation process, the ECG signal, the GSR signal and the PSG signal are acquired, and the feature selection adopts a way combining the random search algorithm and the CfsSubsetEval attribute evaluation method. The appropriate algorithm combination with high accuracy is selected according to different types of the acquired signals.

step 5: performing the machine learning according to the feature parameters set obtained in the step 4, and establishing the depression assessment mathematic model by utilizing the feature parameters set in the machine learning process, wherein the algorithm for the machine learning can selectively utilize one of or a combination of more than one of the following algorithms: the Bayes classifier, the decision tree algorithm, the Adaboost algorithm, the k-Nearest Neighbor, and the support vector machine (SVM). An expression of the depression assessment mathematic model is:

$$Y = {\sum }_{i = 1}^{n} {a}_{i} {y}_{i} ,$$

wherein, Y is an output value of the depression assessment mathematic model, n is the number of selected machine learning algorithm, Yᵢ is output value of the ith algorithm, αᵢ is coefficient of the ith algorithm, and i is positive integer; After the depression assessment mathematic model based on various physiological information is established, the depression level is evaluated by utilizing the output result of the depression assessment mathematic model, and the depression level is divided into five classes: normal, common, light depression, moderate depression and severe depression.

step 6: inputting the result of depression level assessment of the step 5 into the output result module.

The above-mentioned embodiments are preferable embodiments of the present invention, but the embodiments of the present invention are not limited to the above embodiments. Any other alteration, modification, replacement, combination and simplification made without departing from the spiritual essence and principle of the present invention are equivalent replacement ways and shall be incorporated in the protection scope of the present invention.

What is claimed is:

1. A depression assessment system based on the physiological information, comprising: an information acquisition module, a signal processing module, a parameters calculation module, a feature selection module, a machine learning module and an output result module successively connected,
wherein the information acquisition module is used for acquiring electrocardiogram (ECG) signal and one or more of photoplethysmography (PPG) signal, electroencephalogram (EEG) signal, galvanic skin response (GSR)signal, electrogastrography (EGG) signal, electromyogram (EMG) signal, electrooculogram (EOG) signal, polysomnogram (PSG) signal and temperature signal; the signal acquired by the information acquisition module is transmitted in a wire transmission manner bya USB serial port or transmitted in a Bluetooth wireless transmission manner to the signal processing module;
wherein the signal processing module is used for performing the signal processing on the acquired physiological information and comprises an ECG signal processing unit, an PPG signal processing unit, an EEG signal processing unit, an GSR signal processing unit, an EGG signal processing unit, an EMG signal processing unit, an EOG signal processing unit, an PSG signal processing unit and a temperature signal processing unit; the processing of the physiological information comprises baseline removal processing, filtering de-noising processing, heartbeat interval extraction processing, time/frequency transformation processing as well as spectral analysis and spectral estimation processing; and the signal processing module transmits processed signal to the parameters calculation module;
the ECG signal processing unit is used for performing the baseline removal processing, the filtering de-noising processing, extraction of RR intervals processing, interpolation processing, Fourier transformation processing as well as the spectral analysis and the spectral estimation processing;
the PPG signal processing unit is used for performing the baseline removal processing, the filtering de-noising processing, the extraction of PP intervals processing, the interpolation processing, the Fourier transformation processing as well as the spectral analysis and the spectral estimation processing;
the EEG signal processing unit is used for performing the baseline removal processing, threshold value de-noising processing, wavelet decomposition processing as well as the spectral analysis and the spectral estimation processing
the GSR signal processing unit is used for performing the baseline removal processing and wavelet filtering processing;
the EGG signal processing unit is used for performing the baseline removal processing, Hilbert-Huang transformation processing, wavelet analysis processing, multi-resolution analysis processing and independent component analysis processing
the EMG signal processing unit is used for performing the baseline removal processing and wavelet packet self-adaptive threshold value processing;
the EOG signal processing unit is used for performing the baseline removal processing, weighting median filtering processing and wavelet transformation processing;
the PSG signal processing unit is used for processing sleep EEG signal, sleep EMG signal and sleep EOG signal, for performing the baseline removal processing, the threshold value de-noising processing, the wavelet analysis processing as well as the spectral analysis and the spectral estimation processing on the sleep EEG signal, for performing the baseline removal processing, the weighting median filtering processing and the wavelet transformation processing on the sleep EOG signal, and performing the baseline removal processing, the wavelet packet self-adaptive threshold value de-noising processing and the sleep staging processing on the sleep EMG signal;
the temperature signal processing unit is used for performing the baseline removal processing, the threshold value filtering processing, establishment of a relational expression between a temperature value and an image gray value, and the drawing of a heat energy distribution diagram of the human body,
wherein the parameters calculation module is used for calculating the signal parameters of the processed signal comprising time-domain parameters, frequency-domain parameters and time-domain geometric parameters of the heat rate variability, and for calculating the time-domain parameters, the frequency-domain parameters, the histogram parameters and the distribution diagram parameters of one or more of the PPG signal, the EEG signal, the GSR signal, the EGG signal, the EMG signal, the EOG signal, the PSG signal and the temperature signal according to the acquired physiological information,
wherein the feature selection module is used for acquiring the feature parameters set related to the depression level from all signal parameters, and the feature selection module outputs the feature parameters set to the machine learning module,
wherein the machine learning module is used for training depression level quantification classifier and utilizing the feature parameters set to establish the depression assessment mathematic model to quantify the depression level; and the machine learning module inputs the quantified depression level to the output result module,
wherein the output result module is used for displaying the quantified depression level inputted by the machine learning module.

2. The depression assessment system based on the physiological information according to claim 1, wherein the information acquisition module is used for acquiring ECG signal and also used for acquiring one or more physiological information signals of PPG signal, EEG signal, GSR signal, EGG signal, EMG signal, EOG signal, PSG signal and temperature signal; the method of acquiring ECG signal is 3-lead ECG method; in the 3-lead ECG acquiring method, after subjected to amplification, filtering and analog-digital conversion, the acquired ECG signal is transmitted to a computer through data transmission; and the data transmission adopts a wire transmission manner by a USB serial port or a Bluetooth wireless transmission manner.

3. The depression assessment system based on the physiological information according to claim 1, wherein the parameters calculation module comprises an ECG parameters calculation unit, an PPG parameters calculation unit, an EEG parameters calculation unit, an GSR parameters calculation unit, an EGG parameters calculation unit, an EMG parameters calculation unit, an EOG parameters calculation unit, an PSG parameters calculation unit and a temperature parameters calculation unit.

4. The depression assessment system based on the physiological information according to claim 3, wherein the ECG parameters calculation unit comprises the calculation of the RR intervals, the time-domain parameters, the frequency-domain parameters and the time-domain geometric parameters;
the PPG parameters calculation unit comprises the calculation of the RR intervals, the time-domain parameters, the frequency-domain parameters and the time-domain geometric parameters;
the EEG parameters calculation unit is used for calculating δ wave amplitude, δ wave power, δ wave mean value, δ wave variance, δ wave deviation degree, δ wave kurtosis, θ wave amplitude, θ wave power, θ wave mean value, θ wave variance, θ wave deviation, θ wave kurtosis, α wave amplitude, α wave power, α wave mean value, α wave variance, α deviation degree, α wave kurtosis, β wave amplitude, β wave power, β wave mean value, β wave variance, β wave deviation degree, β wave kurtosis and wavelet entropy;
the GSR parameters calculation unit is used for calculating sympathetic skin response latency, the sympathetic skin response amplitude and the skin resistance value;
the EGG parameters calculation unit is used for calculating normogastria, the slow wave, the bradygastria and tachygastria components;
the EMG parameters calculation unit is used for calculating the basic value, the minimum value, the highest value, the EMG decreasing capacity and the EMG curve;
the EOG parameters calculation unit is used for calculating R wave, r wave, S wave and s wave components;
the PSG sleep signal parameters calculation unit is used for calculating sleep latency, total sleep time, arousal index, shallow sleep period (S1), light sleep period (S2), middle sleep period (S3), deep sleep period (S4), rapid eye movement (REM) sleep percentage, REM sleep cycles, REM sleep latency, REM sleep intensity, REM sleep density and REM sleep time; and
the temperature parameters calculation unit is used for calculating the temperature distribution in the human body and drawing the heat energy diagram of the human body.

5. The depression assessment system based on the physiological information according to claim 4, wherein the calculation of the RR intervals in the ECG parameters calculation unit comprises mean value of all RR intervals, standard deviation of NN intervals (SDNN) of heartbeat intervals, root mean square of successive difference( RMSSD) of successive heartbeats, percentage of normal-to-normal interval more than 50 ms (PNN50) of successive heartbeats, standard deviation of successive differences (SDSD) of heartbeats, very low frequency (VLF) power , low frequency (LF) power, high frequency (HF) power, total power (TP), ratio of the low frequency power to the high frequency power (LF/HF), standard deviation (SD1) perpendicular to y=x in RR intervals scatter diagram, standard deviation (SD2) of a y=x straight line in the RR intervals scatter diagram, slope (a1) of the short-term detrended fluctuation analysis and slope (a2) of the long-term detrended fluctuation analysis;
the calculation of the PP intervals in the PPG parameters calculation unit comprises mean value of all PP intervals, standard deviation of NN intervals (SDNN) of pulse intervals, root mean square of successive difference (RMSSD) of successive pulses, percentage of normal-to-normal interval more than 50 ms (PNN50) of successive pulses, standard deviation of successive differences (SDSD) of pulses, very low frequency (VLF) power, low frequency (LF) power, high frequency (HF) power, total power (TP), ratio of the low frequency power to the high frequency (LF/HF) power, standard deviation (SD1) perpendicular to y=x in PP interval scatter diagram, standard deviation (SD2) of a y=x straight line in the PP interval scatter diagram, slope (a1) of the short-term detrended fluctuation analysis and slope (a2) of the long-term detrended fluctuation analysis; and
in the ECG parameters calculation unit and the PPG parameters calculation unit, the time-domain parameters comprise mean value, SDNN, RMSSD, PNN50 and SDSD; the frequency-domain parameters comprise VLF, LF, HF, TP and LF/HF; the time-domain geometric parameters comprise SD1, SD2, a1 and a2.

6. An assessment method applied to the depression assessment system based on the physiological information, comprising the steps of:
a) acquiring the physiological information; the physiological information including ECG information, and one or more information of PPG, EEG, GSR, EGG, EMG, EOG, PSG and temperature,
b) processing the acquired signals such as the ECG signal and one or more of the PPG signal, the EEG signal, the GSR signal, the EGG signal, the EMG signal, the EOG signal, the PSG signal and the temperature signal,
c) calculating the processed signal to obtain signal parameters;
d) normalizing the calculated signal parameters, and performing the feature selection on parameters set formed by the normalized signal parameters to obtain feature parameters set;
e) performing the machine learning by utilizing the feature parameters set obtained in step d), establishing a depression assessment mathematic model by utilizing the relationship between the feature parameters set and the depression level, outputting a depression level assessment result by utilizing the depression assessment mathematic model, and assessing the depression level according to the depression level assessment result;
the machine learning being used for training the depression assessment mathematic model, establishing the depression assessment mathematic model by utilizing the feature parameters set during the machine learning process, and utilizing one of or a combination of more than one of the following algorithms for the machine learning algorithm: bayes classifier, decision tree algorithm, AdaBoost algorithm, k-nearest-neighbor algorithm and support vector machine; expression of the depression assessment mathematic model is as follows:
$$Y = {\sum }_{i = 1}^{n} {a}_{i} {y}_{i}$$
wherein, Y is an output value of the depression assessment mathematic model, n is the number of selected machine learning algorithm, Yᵢ is output value of the ith algorithm, αᵢ is coefficient of the ith algorithm, and i is positive integer;
f) inputting the result of depression level assessment of the step e) into the output result module;
in the step c), the calculation of signal parameters of the processed signal includes the ECG parameters calculation, the PPG parameters calculation, the EEG parameters calculation, the GSR parameters calculation, the EGG parameters calculation, the EMG parameters calculation, the EOG parameters calculation, the PSG parameters calculation and the temperature parameters calculation; the ECG parameters calculation includes the calculation of the RR intervals, the time-domain parameters, the frequency-domain parameters and the time-domain geometric parameters; the time-domain parameters include mean value, SDNN, RMSSD, PNN50 and SDSD; the frequency-domain parameters include VLF, LF, HF, TP and LF/HF; the time-domain geometric parameters include SD1, SD2, a1 and a2; the PPG parameters calculation includes the calculation of the PP intervals, the time-domain parameters; the time-domain parameters include mean value, SDNN, RMSSD, PNN50 and SDSD; the frequency-domain parameters include VLF, LF, HF, TP and LF/HF; the time-domain geometric parameters include SD1, SD2, a1 and a2; the EEG parameters calculation includes the calculation of δ wave amplitude, δ wave power, δ wave mean value, δ wave variance, δ wave deviation degree, δ wave kurtosis, θ wave amplitude, θ wave power, θ wave mean value, θ wave variance, θ wave deviation, θ wave kurtosis, α wave amplitude, α wave power, α wave mean value, α wave variance, α deviation degree, α wave kurtosis, β wave amplitude, β wave power, β wave mean value, β wave variance, β wave deviation degree, β wave kurtosis and wavelet entropy; the GSR parameters calculation includes the calculation of sympathetic skin response latency, the sympathetic skin response amplitude and the skin resistance value; the EGG parameters calculation includes the calculation of normogastria, the slow wave, the bradygastria and tachygastria components; the EMG parameters calculation includes the calculation of basic value, the minimum value, the highest value, the EMG decreasing capacity and the EMG curve; the EOG parameters calculation includes the calculation of R wave, r wave, S wave and s wave components; the PSG sleep signal parameters calculation includes the calculation of the sleep latency, the total sleep time, the arousal index, S1, S2, S3, S4, the REM sleep percentage, the REM sleep cycles, the REM sleep latency, the REM sleep intensity, the REM sleep density and the REM sleep time; and the temperature parameters calculation includes the calculation of the temperature distribution in the human body.

7. The assessment method for the depression assessment system based on the physiological information according to claim 6, wherein in step d), the normalizing method is:
$${X}_{in} = \frac{{X}_{i} - {X}_{imean}}{{X}_{istd}} ,$$
wherein, X refers to signal parameter of the parameter set; Xᵢ indicates the ith normalized signal parameter value, Xᵢₙ indicates the ith normalized value, Xᵢₘₑₐₙ indicates normal mean value of the ith parameter, X_{istd} indicates normal standard difference of the ith parameter, and i is positive integer.

8. The assessment method for the depression assessment system based on the physiological information according to claim 6, wherein in the step b), the signal processing includes the ECG signal processing, the PPG signal processing, the EEG signal processing, the GSR signal processing, the EGG signal processing, the EMG signal processing, the EOG signal processing, the PSG signal processing and the temperature signal processing; the ECG signal processing includes the baseline removal processing, the filtering de-noising processing, the RR intervals extraction, the interpolation processing, the Fourier transformation processing as well as the spectral analysis and spectral estimation processing; the EEG signal processing includes the baseline removal processing, the threshold value de-noising processing, the wavelet decomposition processing as well as the spectral analysis and spectral estimation processing; the GSRsignal processing includes the baseline removal processing and the wavelet filtering processing; the EGG signal processing includes the baseline removal processing, the Hilbert-Huang transformation processing, the wavelet analysis, the multi-resolution analysis and the independent component analysis; the EMG signal processing includes the baseline removal processing and the wavelet packet self-adaptive threshold value de-noising processing; the EOG signal processing includes the baseline removal processing, the weighting median filtering processing and the wavelet transformation processing; the PSG signal processing includes the processing of the sleep EEG signal, the sleep EMG signal and the sleep EOG signal; the baseline removal processing, the threshold value de-noising processing, the wavelet decomposition processing as well as the spectral analysis and spectral estimation processing are conducted on the sleep EEG signal; the baseline removal processing, the weighted median filtering processing and the wavelet transformation processing are conducted on the sleep EOG signal; the baseline removal processing, the wavelet packet self-adaptive threshold value de-noising processing and the sleep staging processing are conducted on the sleep EMG signal; and the temperature signal processing includes the baseline removal processing, the threshold value filtering processing and the establishment of a relational expression between the temperature value and the image gray value.

9. The assessment method for the depression assessment system based on the physiological information according to claim 6, wherein in the step d), the feature selection trains a data set according to all signal parameters outputted by the parameters calculation module, each sample is represented by a feature set, and a feature sub-set is generated; an optimum feature subset in the feature set is acquired in a searching manner according to the evaluation criteria; the current feature subsets are compared and evaluated; when the acquired feature subset is the optimum feature subset, a termination condition is satisfied, and the feature parameters set related to the depression level is outputted; the search algorithm adopts one of or a combination of more than one of the following algorithms: the complete search algorithm, the sequential search algorithm, the random search algorithm, the genetic algorithm, the simulated annealing search algorithm and the traceable greedy search expansion algorithm; and the evaluation criteria adopts one of or a combination of two of the following algorithms: the wapper model and the CfsSubsetEval attribute assessment method.
